(19) United States
(12) Patent Application Publication
(10) Pub. No.: US 2022/0054054 A1
(43) Pub. Date: Feb. 24, 2022

(54) System for Determining Treatment Decisions for Control of Blood Glucose

(71) Applicant: QUIN TECHNOLOGY LTD, London (GB)

(72) Inventors: Isabella Elisabeth Degen, London (GB); Vyvyan Codd, London (GB); Zareen Ali, London (GB); Nelia Kleiven, London (GB)

(21) Appl. No.: 17/416,823

(22) PCT Filed: Nov. 14, 2019
(86) PCT No.: PCT/GB2019/053228

§ 371 Date: Jun. 21, 2021

(30) Foreign Application Priority Data
Dec. 21, 2018 (GB) 1821091.4

(51) Int. Cl. A61B 5/145; G16H 10/60; G16H 20/10

(52) U.S. Cl. CPC A61B 5/14532; G16H 20/10; G16H 10/60

(57) ABSTRACT

A system for determining treatment decisions for control of blood glucose in a patient, the system comprising: a processor; and a memory; wherein, the memory stores a personal history of glycaemic change mediating factors experienced by the patient, the personal history comprising: a current situation of glycaemic change mediating factors relevant to the patient; and a record of glycaemic change mediating factors experienced by the patient in historical situations at times before the current situation; and wherein the processor is arranged, when determining a treatment decision for control of blood glucose in the patient: to compare the patient's current situation with the historical situations, to thereby identify a historical situation that corresponds to the current situation; and in response to identifying a historical situation that corresponds to the current situation, to determine a glycaemic change mediating factor to be applied to maintain the patient's future blood glucose level within an allowable range, based on the identified historical situation.

### BACKGROUND

### Technical Field

The present invention relates to systems for determining treatment decisions for control of blood glucose in a patient. The present invention also relates to methods and computer program products in this field.

### Description of Related Art

Management of blood glucose is an ongoing challenge for people that have diabetes. Taking of blood samples to regularly measure blood sugar levels gives people feedback on how well the balance between consumption of food, which tends to increase blood glucose, and the administration of pharmacologically active agents, for example insulin or synthetic insulin analogues which tend to reduce blood glucose, is controlling blood glucose to remain within an allowable range. However, for many people, maintaining their blood glucose levels within a desired range is still a challenge as they go about their day to day life, given the complicated relationship between glycaemia and administration of pharmacologically active agents, food consumption and other factors such as activity level, the environment and each individual's metabolic and endocrine system.

Recording a history of blood glucose measurements and administration of pharmacologically active agents over time is known, as is apparatus for displaying such a recorded history. However, these known techniques and apparatus are not that useful in guiding decision making on the nature and timing of the most suitable next actions in management of glycaemia, since they do not reflect the expected impact of recent actions undertaken by the patient, factors which are not under the direct/conscious control of a patient, nor expected future actions or conditions.

Example embodiments aim to address problems associated with existing related solutions, whether specifically mentioned above or which can otherwise be appreciated from the discussion herein.

### SUMMARY OF THE INVENTION

In one aspect there is provided a system for determining treatment decisions for control of blood glucose in a patient, the system comprising: a processor; and a memory; wherein, the memory stores a personal history of glycaemic change mediating factors experienced by the patient, the personal history comprising:

a current situation of glycaemic change mediating factors relevant to the patient; and a record of glycaemic change mediating factors experienced by the patient in historical situations at times before the current situation; and wherein the processor is arranged, when determining a treatment decision for control of blood glucose in the patient:

to compare the patient's current situation with the historical situations, to thereby identify a historical situation that corresponds to the current situation; and

in response to identifying a historical situation that corresponds to the current situation, to determine a glycaemic change mediating factor to be applied to maintain the patient's future blood glucose level within an allowable range, based on the identified historical situation.

In one example, the processor is arranged, when determining a treatment decision for control of blood glucose in the patient: to compare the patient's current situation with the historical situations, to thereby identify a plurality of historical situations that correspond to the current situation; and in response to identifying a plurality of historical situations that correspond to the current situation, to determine a glycaemic change mediating factor to be applied to maintain the patient's future blood glucose level within an allowable range, based on the identified historical situations.

In one example the memory stores a personal history of glycaemic change mediating factors experienced by the patient, the personal history comprising: a record of glycaemic change mediating factors experienced by the patient in historical situations at times before the current situation together with associated blood glucose level outcomes for the patient after the historical situations.

In one example, the processor is arranged, when determining a treatment decision, to compare the patient's current situation with those of the historical situations, to identify one or more historical situations corresponding to the current situation; and in response to identifying one or more corresponding historical situations to determine a glycaemic change mediating factor to be applied to maintain the patient's future blood glucose level within an allowable range, by reference to a blood glucose level outcome of the one or more corresponding historical situations in which the patient's blood glucose level was maintained in the allowable range after the historical situation.

In one example the memory stores a personal history of glycaemic change mediating factors experienced by the patient, comprising the current situation of glycaemic change mediating factors relevant to the patient, and the patient's current blood glucose level.

In one example the memory stores a personal history of glycaemic change mediating factors experienced by the patient, comprising the current situation of glycaemic change mediating factors relevant to the patient, along with the patient's expected future glycaemic change mediating factors.

In one example the memory is arranged to store a personal history of glycaemic change mediating factors experienced by the patient, the glycaemic change mediating factors included in a group comprising: administration of a pharmacologically active agent, consumption of food, performance of physical activity, heart rate, patient weight, body temperature, exposure to external environmental stimulus; and hormonal factors.

In one example the processor is arranged to determine a glycaemic change mediating factor to be applied to maintain the patient's future blood glucose level within an allowable range, the determined glycaemic change mediating factor selected from a group comprising: administration of a pharmacologically active agent, consumption of food, performance of physical activity, the glycaemic change mediating factor to be applied either alone or in combination with others from said group.

In one example the system comprises a manual data input unit operable to receive manual input of data relating to glycaemic change mediating factors experienced by the patient and/or to receive manual input of data relating to the patient's blood glucose level, and to provide such data received by the manual data input unit to the memory for storage in the personal history of glycaemic change mediating factors experienced by the patient.

In one example the manual data input unit is operable to receive manual data input of data relating to the patient's expected future glycaemic change mediating factors, and to provide such data received by the manual data input unit to the memory for storage as part of the current situation of glycaemic change mediating factors relevant to the patient.

In one example the system comprises a data logging unit arranged to automatically log data relating to glycaemic change mediating factors experienced by the patient, and to provide the automatically logged data to the memory for storage in the personal history of glycaemic change mediating factors experienced by the patient.

In one example the system comprises an output unit arranged to provide information to the patient relating to one or more historical situations identified by the processor as historical situations that correspond to the current situation.

In one example the system is formed as a portable electronic device. In one example the system is provided via distributed computing environment.

In accordance with another aspect, there is provided a method of determining treatment decisions for control of blood glucose in a patient, the method comprising:

storing a personal history of glycaemic change mediating factors experienced by the patient, the personal history comprising: a current situation of glycaemic change mediating factors relevant to the patient; and a record of glycaemic change mediating factors experienced by the patient in historical situations at times before the current situation;

comparing the patient's current situation with the historical situations, to thereby to identify historical situation that corresponds to the current situation; and

in response to identifying a historical situation that corresponds to the current situation, to determine a glycaemic change mediating factor to be applied to maintain the patient's future blood glucose level within an allowable range, based on the identified historical situation.

In another aspect there is provided a tangible non-transient computer-readable storage medium having recorded thereon instructions which, when implemented by a computer system, cause the computer system to carry out the steps of the method set out above.

According to other aspects there are provided systems, methods, and computer program products as set forth in the appended claims. Other features of the invention will be apparent from the dependent claims, and the description which follows.

### BRIEF DESCRIPTION OF THE DRAWINGS

For a better understanding of the invention, and to show how example embodiments may be carried into effect, reference will now be made to the accompanying drawings in which:

FIG. 1 is a schematic view of a system in accordance with an example embodiment;

FIG. 2 is an example screen shot illustrating a current situation of glycaemic change mediating factors;

FIG. 3 is an example screen shot illustrating a historical situation of glycaemic change mediating factors;

FIGS. 4a and 4b are example screen shots illustrating the output of a method of rendering data representing the effect of a glycaemic change mediating factors on glycaemia;

FIG. 5 is an example screen shot illustrating another historical situation of glycaemic change mediating factors;

FIG. 6 is flowchart illustrating an example method according to an example embodiment; and

FIG. 7 shows a computer-readable storage medium.

### DETAILED DESCRIPTION OF THE EXAMPLE EMBODIMENTS

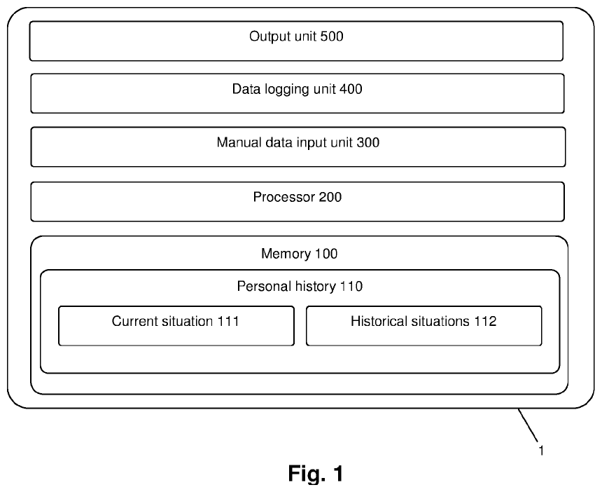

FIG. 1 is a schematic view of an example system 1 for determining treatment decisions for control of blood glucose in a patient, the system 1 comprising a portable electronic device. The system 1 comprises a memory 100, a processor 200, a manual data input unit 300, a data logging unit 400 and an output unit 500. The components of the system 1 are mutually interconnected for data transmission therebetween, for example over a bus (not shown). The system 1 may be implemented as a smartphone, but may equally be implemented as a portable computing device, laptop, tablet, wearable device, or an emulated virtual device on any appropriate host hardware and may have its functionality provided in part by data storage or processing capacity accessed over a data network in a client-server architecture.

The system 1 suitably comprises an application program loaded on the processor 200 that controls the processor 200 to enable the system 1 to collect information relevant to glycaemia, via the manual data input unit 300 and the data logging unit 400, and which is configured to undertake operations thereafter which enable the system 1 to provide information to a patient that is helpful for controlling changes in glycaemia, in particular by making a suggestion of one or more glycaemic change mediating factors that the patient should consider applying, according to a current situation.

The memory 100 stores a personal history 110 of glycaemic change mediating factors experienced by the patient, the personal history comprising data of a current situation 111 and data of historical situation 112. The data of the current situation 111 represents the glycaemic change mediating factors experienced by the patient and which are relevant to the patient's glycaemia at the current time. Data of the current situation 111 may be data that is manually input via the manual data input unit 300 and/or data that is automatically recorded by the data logging unit 400.

The data stored in the personal history 110 as data of historical situations 112 is data of previous current situations, i.e. situations which are no longer relevant to the patient's blood glucose level, because the mediating factors impacting blood glucose when that situation was a current situation are no longer relevant.

The processor 200 is arranged to perform a process of determining a treatment decision for control of blood glucose in the patient by comparing the data of a current situation 111 with data of the historical situations 112. Based on this comparison the processor 200 identifies a historical situation that corresponds to the current situation. Once a corresponding historical situation has been identified, the processor 200 determines a glycaemic change mediating factor to be applied in order to maintain the patient's future blood glucose level within an allowable range,. The determined glycaemic change mediating factor to be applied in order to maintain the patient's future blood glucose level can be provided to the output unit 500 for rendering, so that in response the patient may take action in response thereto. In order to aid the comparison between a current situation and historical situations, example embodiments may also include a current blood glucose measurement as data of the current situation 111, and may include blood glucose measurements obtained in the past as data of the historical situations.

In this way, the data of historical situations 112 is used to give a patient-specific recommendation for consumption of food, administration of a pharmacologically active agent or other glycaemic change mediating factor that can be expected to produce a similar result, or improved result, in terms of blood glucose control.

As will be appreciated, the greater the number of historical situations whose data is stored in the personal history 110 the more likelihood there is of closely corresponding situation being found in the data of historical situations 112, and once the current situation is determined to be over the processor is arranged to store the data of the current situation 111 as data of a historical situation 112 for use in future comparisons.

The manual data input unit 300 receives input from a patient regarding glycaemic change mediating factors that have been experienced by the patient, for example data concerning administration of a pharmacologically active agent such as insulin or a synthetic insulin analogue. This is associated with a time of administration, what active agent, and in what dosage. Other data regarding glycaemic change mediating factors that have been experienced by the patient that can be input in this way includes, for example, data regarding consumption of food, performance of physical activity, patient weight, age, body temperature, exposure to external environmental stimulus, hormonal factors and the like, all of which are known to have an effect on glycaemia. Again this data may be include information on what has been experienced, including an amount, duration or similar quantifiable measure relating to a glycaemic change mediating factor, and a commencement time or duration. The inputs received by the manual data input unit 300 may relate to previously experienced factors, associated with a commencement time in the past, and/or may relate to factors that the patient knows they will experience in the future. For example, the patient may be about to eat some food; this will have an impact on the patient's glycaemia which is relevant to the current situation and glycaemic change mediating factor to be applied to maintain the patient's future blood glucose level within an allowable range.

In addition, or independently of manual data inputs, the data of the historical and current situations may be compiled and stored based on data that is automatically recorded by the data logging unit 400. Certain information which is relevant to glycaemic, but which is difficult to record manually may be obtained using the data logging unit 400 using sensors thereof or external sensors (not shown) that communicate with the system 1. For example, the data logging unit 400 may comprise, or be coupled to, a heart rate monitor to log a continuous record of patient heart rate. Similarly, the data logging unit 400 may operate to automatically generate data relating to predictable according to known date/time information, such as information relating to circadian rhythms.

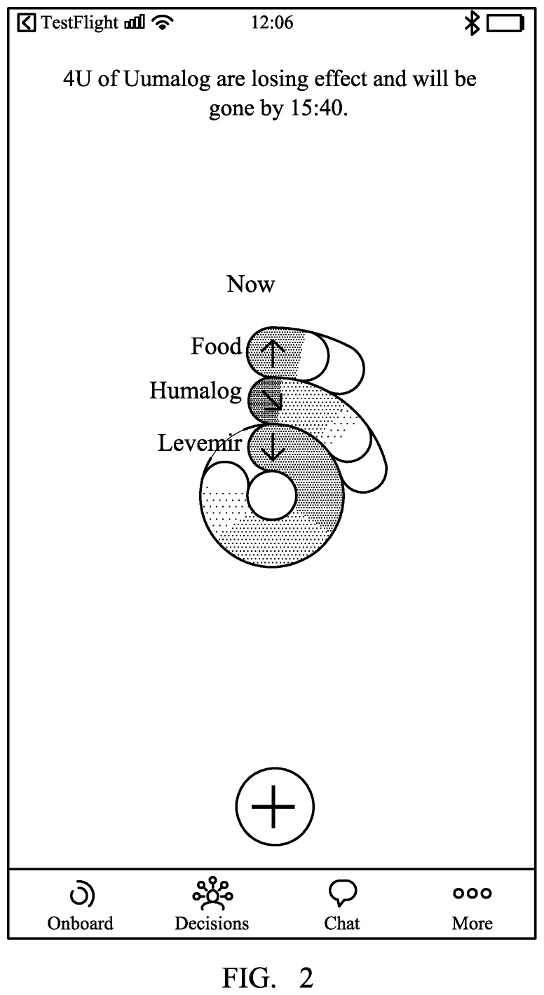

FIG. 2 shows an example screen shot illustrating a current situation of glycaemic change mediating factors. In green, indicating a factor that serves to raise blood glucose, is a timeline indicating consumption of food and labelled as “Food”. In this timeline the upward effect is illustrated as a strong effect by a vertically upward arrow, and the fade out of the timeline indicates a diminishing of the effect over time. In blue, indicating a factor that serves to lower blood glucose, is a timeline indicating administration of a pharmacological agent and labelled as “Humalog”. In this timeline a downward effect is illustrated as a weak effect by a diagonally downward arrow, and the fade out of the timeline indicates a diminishing effect over time. Also in blue, indicating a factor that serves to lower blood glucose, is a timeline indicating administration of a pharmacological agent and labelled as “Levemir”. In this timeline a downward effect is illustrated as a strong effect by a vertically downward arrow, and the fade out of the timeline indicates a diminishing effect over time. The twelve o′clock position corresponds to the current time, and the timelines are scaled to one another according to the expected effect of the factors. The memory 100 suitably includes conversion factors that enable the processor 200 to relate the data of the current situation 111, corresponding to data input through the manual data input by the patient regarding the food consumption (type of food, amount of food and time of consumption) to the type of output shown in FIG. 2 and for use in comparison between data of a current situation 111 with data of historical situations 112. Corresponding conversion factors are suitably included for other glycaemic change mediating factors.

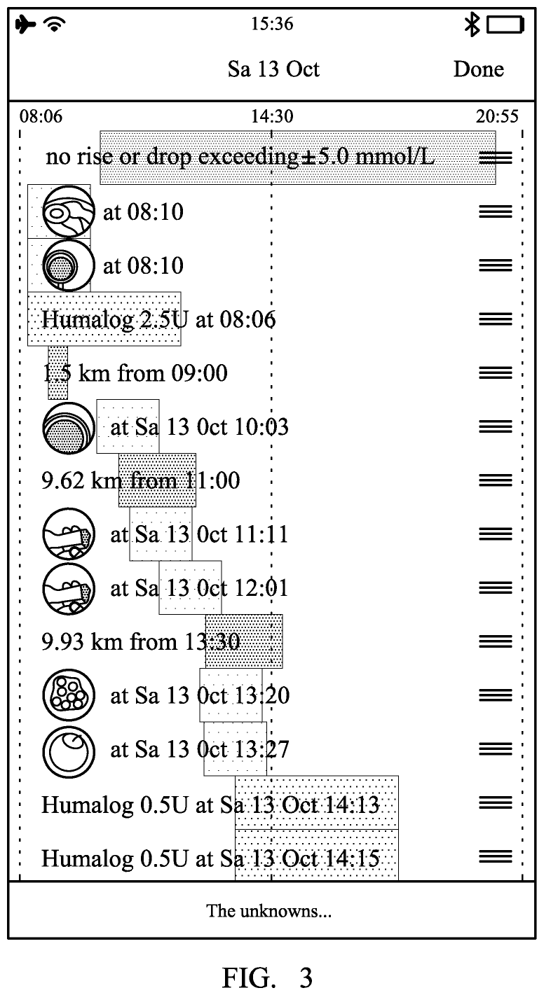

FIG. 3 is an example screen shot illustrating a historical situation of glycaemic change mediating factors, corresponding to the data stored as data of a historical situation 112 in the personal history 110. As will be appreciated, although this representation is described with reference to a historical situation, the situation would have been a current situation at an earlier point in time, with data input using the manual data input unit 300 or from the data logging unit 400. FIG. 3 shows, using the same colour coding of green timelines for food, and blue timelines for pharmacologically active agents, factors experienced by the patient, commencement times and information about what was consumed/administered. The timelines indicate the extent of the effect of each factor on glycaemia, over time. In addition, yellow timelines are shown which correspond to physical activities performed by the patient. Here the physical activities are represented on a distance walked.

A detailed breakdown of the factors experienced by the patient, in this example situation, is given below:

8.10: coffee, bacon, eggs+insulin at 8.06

9.00: walk to the tube station, travel to Henley-on-Thames

10.00: drink a latte

11.00: start a long walk

11.11: hypo+hypo treatment

12.01: hypo+hypo treatment

13.00: break for lunch

13.30: continue walking

14.13: take 0.5U insulin

14.14: take another 0.5U

There is overlap between the timelines, such that at any given time at least two factors were having an effect of blood glucose of the patient, meaning that when undertaking the comparison between the data relativity to the situations, the processor 200 is able to determine this set of factors correspond to a single situation. By storing data of this nature in the personal history 110, and applying suitable matching algorithms based on degree of similarity, it is possible for the processor 300 to make valid comparisons between the current situation and historical situations to determine effective choices that the patient can make to maintain their blood glucose in an allowable range, for example between predetermined maximum and minimum values. The pink timeline at the top of the image shows the range of blood glucose variation experienced during this situation.

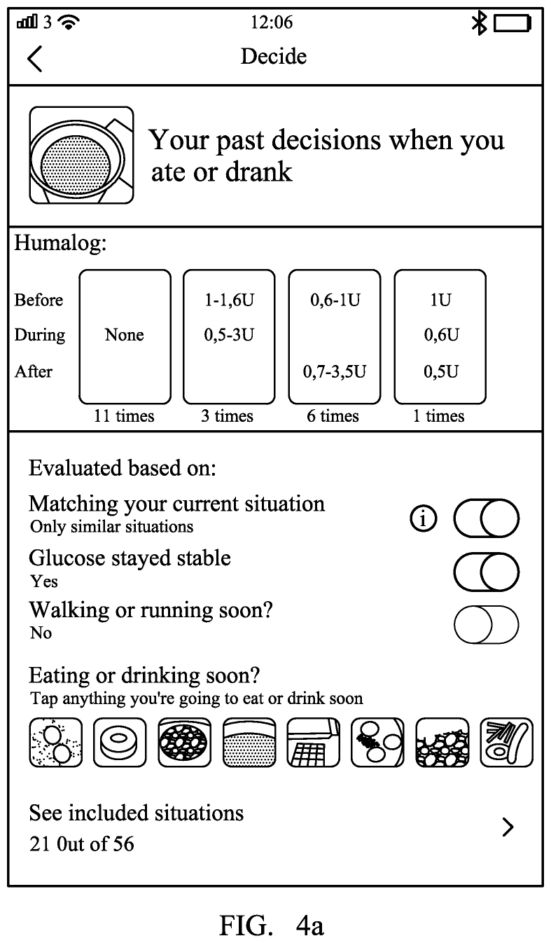

FIGS. 4a and 4b are example screen shots illustrating the output of a method of rendering data representing the effect of a glycaemic change mediating factors on glycaemia. In FIG. 4a the patient is ready to consume food, in this case a latte as represented by the image in the top left. The patient has input data to this effect, which forms part of the current situation as an expected future action. The processor 200 has compared data of the current situation with that of historical situations, and identified twenty one out of fifty six as potentially relevant. The included situations in this example are those in which it was determined that the patient's blood glucose remained stable, within an allowable range. The patient is provided the result of the determination by the processor 200, in terms of the eleven times that historical situations the administration of no Humalog resulted in the blood glucose remaining in the allowable range, the three times that 1-1.6U were administered before and 0.5-3U were administered during, and so.

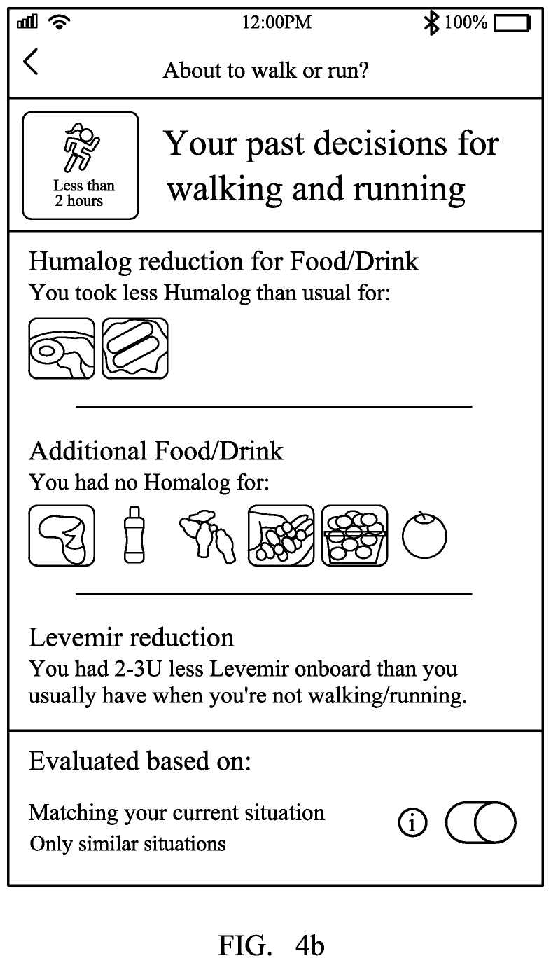

The patient can then determine which mediating factor to apply, based on the determination of the processor 200. The patient is prompted to add any other food items that may be consumed soon, and is given the option to see the corresponding situations in more detail, as will be explained in more detail with reference to FIG. 5.

FIG. 4b generally corresponds to FIG. 4a, but in this instance the patient is ready to perform physical activity. The information on similar situations is provided in order of which of the historical situations are determined to be the most closely matched to the current situation.

FIG. 5 is an example screen shot illustrating another historical situation of glycaemic change mediating factors, this time presented as three of the matching situations identified as per FIG. 4b. The timelines of activity (yellow), food (green) and pharmacologically active agent (blue) are aligned according to the start of the physical exercise, time and date information provided and an indication of what food was consumed. Any out of range information on blood glucose in these situations can also be provided, but in this example the pink timelines indicate that no major swings in blood glucose were experienced.

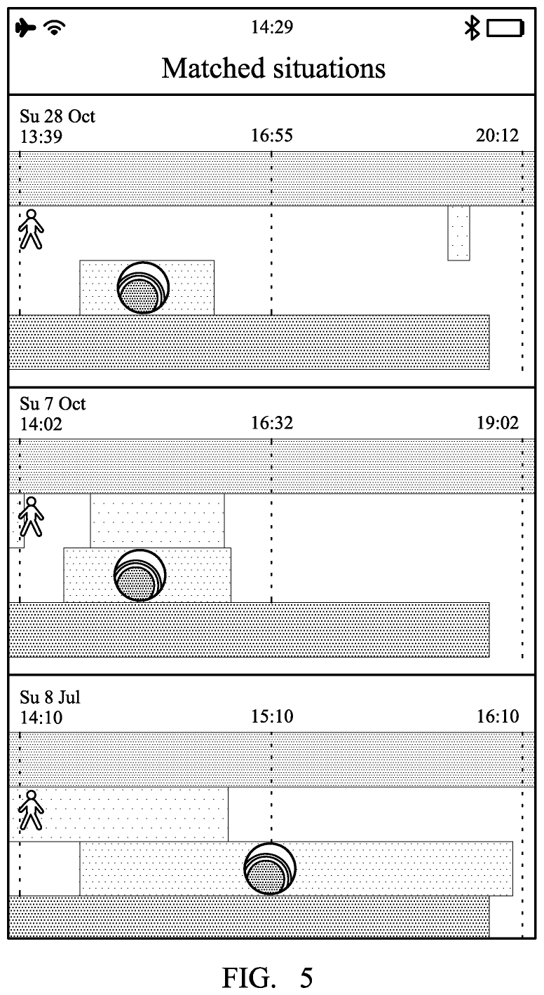

FIG. 6 shows, schematically, a schematic method of determining treatment decisions for control of blood glucose in a patient. The method comprises storing a personal history of glycaemic change mediating factors experienced by the patient, at step S101. The personal history comprises a current situation of glycaemic change mediating factors relevant to the patient; and a record of glycaemic change mediating factors experienced by the patient in historical situations at times before the current situation. At step S102 the method comprises comparing the patient's current situation with the historical situations, to thereby to identify historical situation that corresponds to the current situation. In response to identifying a historical situation that corresponds to the current situation the method comprises at step S103 determining a glycaemic change mediating factor to be applied to maintain the patient's future blood glucose level within an allowable range, based on the identified historical situation. The method may further comprise an optional step, S104, of rendering information on the determined mediating factor for output.

At least some elements discussed herein may be constructed, partially or wholly, using dedicated special-purpose hardware. Terms such as ‘component’, ‘module’ or ‘unit’ used herein may include, but are not limited to, a hardware device, such as a Field Programmable Gate Array (FPGA) or Application Specific Integrated Circuit (ASIC), which performs certain tasks.

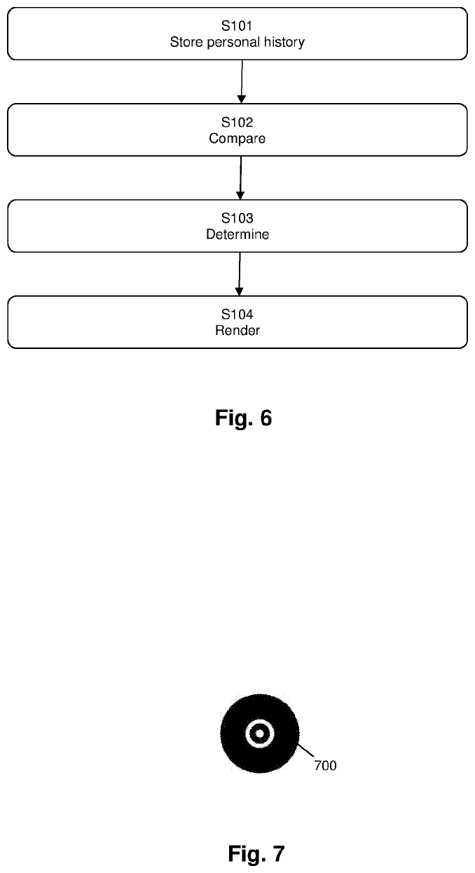

At least some elements may be may be configured to reside on an addressable storage medium and be configured to execute on one or more processors. That is, the elements may be implemented in the form of a tangible computer-readable storage medium having recorded thereon instructions that are, in use, executed by a computer or other suitable device. FIG. 7 shows such a medium 700. The elements may include, by way of example, components such as software components, object-oriented software components, class components and task components, processes, functions, attributes, procedures, subroutines, segments of program code, drivers, firmware, microcode, databases, data structures, tables, arrays, and variables. The tangible medium may take any suitable form, but examples include solid-state memory devices (ROM, RAM, EPROM, EEPROM, etc.), optical discs (e.g. Compact Discs, DVDs, and others), magnetic discs, magnetic tapes and magneto-optic storage devices.

The example embodiments have been described with reference to the example components, modules and units discussed herein. Where appropriate, these functional elements may be combined into fewer elements or separated into additional elements. In some cases, the elements are distributed over a plurality of separate computing devices that are coupled by a suitable communications network, including any suitable wired networks or wireless networks. Throughout this specification, the term “comprising” or “comprises” may mean including the component(s) specified but is not intended to exclude the presence of other components.

As above, example embodiments enable a patient to take better decisions on how to apply glycaemic change mediating factors, to ensure that their blood glucose levels are maintained within a predetermined range when considering the current situation of glycaemic change mediating factors, and expected future factors that will be experienced. The use of a personal history of historical situations that can be compared to the current situation means that patient-specific recommendations can be made, improving glucose control beyond a prescribed dosage regimen for pharmacologically active agents that is not responsive to the patient's current situation and which does not take into account how the patient has reacted to glycaemic change mediating factors in the past.

Although a few preferred embodiments have been shown and described, it will be appreciated by those skilled in the art that various changes and modifications might be made without departing from the scope of the invention, as defined in the appended claims.

1-15. (canceled)

16. A system for determining treatment decisions for control of blood glucose in a patient, the system comprising:
a memory configured to store a personal history of glycaemic change mediating factors experienced by the patient, the personal history comprising:
a current situation of glycaemic change mediating factors relevant to the patient; and
a record of glycaemic change mediating factors experienced by the patient in historical situations at times before the current situation;
a processor that, when determining a treatment decision for control of blood glucose in the patient, is configured to:
compare the current situation with the historical situations, to thereby identify a historical situation that corresponds to the current situation; and
in response to identifying a historical situation that corresponds to the current situation, determine a glycaemic change mediating factor to be applied to maintain a future blood glucose level of the patient within an allowable range, based on the identified historical situation.

17. The system of claim 16, wherein the processor, when determining a treatment decision for control of blood glucose in the patient, is further configured to:
compare the current situation with the historical situations to thereby identify a plurality of historical situations that correspond to the current situation; and
in response to identifying the plurality of historical situations that correspond to the current situation, determine the glycaemic change mediating factor to be applied to maintain the future blood glucose level within the allowable range based on the identified historical situations.

18. The system of claim 16, wherein the personal history comprises the record of glycaemic change mediating factors experienced by the patient in historical situations at times before the current situation together with associated blood glucose level outcomes for the patient after the historical situations.

19. The system of claim 18, wherein the processor is configured to determine the glycaemic change mediating factor to be applied to maintain the future blood glucose level within the allowable range by reference to a blood glucose level outcome of one or more historical situations, after the historical situation, in which the patient's blood glucose level was maintained in the allowable range.

20. The system of claim 16, wherein the memory is further configured to store a current blood glucose level of the patient.

21. The system of claim 16, wherein the memory is further configured to store the current situation of glycaemic change mediating factors relevant to the patient along with expected future glycaemic change mediating factors of the patient.

22. The system of claim 16, wherein the glycaemic change mediating factors are comprised in a group comprising administration of a pharmacologically active agent, consumption of food, performance of physical activity, heart rate, patient weight, body temperature, exposure to external environmental stimulus, and hormonal factors.

23. The system of claim 16, wherein to determine the glycaemic change mediating factor to be applied to maintain the future blood glucose level within the allowable range, the processor is configured to select the determined glycaemic change mediating factor from a group comprising administration of a pharmacologically active agent, consumption of food, and performance of physical activity, wherein the glycaemic change mediating factor is to be applied either alone or in combination with one or more others from the group.

24. The system of claim 16, further comprising manual data input circuitry configured to:
receive manual input of data relating to:
the patient's blood glucose level; and/or
glycaemic change mediating factors experienced by the patient; and
provide the data to the memory for storage in the personal history of glycaemic change mediating factors experienced by the patient.

25. The system of claim 16, further comprising manual data input circuitry configured to:
receive manual input of data relating to expected future glycaemic change mediating factors of the patient; and
provide the data to the memory for storage as part of the current situation of glycaemic change mediating factors relevant to the patient.

26. The system of claim 16, further comprising data logging circuitry configured to:
automatically log data relating to glycaemic change mediating factors experienced by the patient; and
provide the automatically logged data to the memory for storage in the personal history of glycaemic change mediating factors experienced by the patient.

27. The system of claim 16, further comprising output circuitry configured to provide information to the patient relating to the historical situation identified by the processor as corresponding to the current situation.

28. The system of claim 16, wherein the system is a portable electronic device.

29. A method of determining treatment decisions for control of blood glucose in a patient, the method comprising:
storing a personal history of glycaemic change mediating factors experienced by the patient, the personal history comprising:
a current situation of glycaemic change mediating factors relevant to the patient; and
a record of glycaemic change mediating factors experienced by the patient in historical situations at times before the current situation;
comparing the current situation with the historical situations, to thereby to identify a historical situation that corresponds to the current situation; and
in response to identifying the historical situation that corresponds to the current situation, determining a glycaemic change mediating factor to be applied to maintain a future blood glucose level of the patient within an allowable range based on the identified historical situation.

30. A non-transitory computer-readable storage medium having recorded thereon instructions which, when executed by a computer system, cause the computer system to:
store a personal history of glycaemic change mediating factors experienced by a patient, the personal history comprising:
a current situation of glycaemic change mediating factors relevant to the patient; and
a record of glycaemic change mediating factors experienced by the patient in historical situations at times before the current situation;
compare the current situation with the historical situations, to thereby to identify a historical situation that corresponds to the current situation; and
in response to identifying the historical situation that corresponds to the current situation, determine a glycaemic change mediating factor to be applied to maintain a future blood glucose level of the patient within an allowable range based on the identified historical situation.
